(19) United States
(12) Patent Application Publication
(10) Pub. No.: US 2015/0262434 A1
(43) Pub. Date: Sep. 17, 2015

(54) PERFORMANCE OF EXPERIMENTS

(71) Applicant: FEV GmbH, Aachen (DE)

(72) Inventors: Thorsten Schnorbus, Aachen (DE); Joschka Schaub, Aachen (DE)

(73) Assignee: FEV GMBH, Aachen (DE)

(21) Appl. No.: 14/416,515

(22) PCT Filed: Jul. 23, 2013
(86) PCT No.: PCT/EP2013/002174

§ 371 Date: Apr. 10, 2015

(30) Foreign Application Priority Data
Jul. 23, 2012 (DE) 10 2012 014 469.5

(51) Int. Cl. G07C 5/08; G01N 33/00; G01M 15/10

(52) U.S. Cl. CPC G07C 5/08; G01M 15/102; G01N 33/0037; G01N 33/004

(57) ABSTRACT

The invention relates to the creation of an experimental plan and the performance of a series of measurements, comprising the determination of operating data of a drive device to he tested and belonging to a vehicle by means of an automated statistical experimental plan (DoE), wherein the experimental plan includes at least the following steps: identification of one or more target variables which the drive device has to meet during test operation, and narrowing relevant values to the one or more target variables, assignment of one or more actuating variables to the drive device with one or more target variables and automated creation of the experimental plan on the basis of at least two target variables to be met.

### CROSS-REFERENCE TO RELATED APPLICATIONS

This application is the U.S. national phase of PCT/EP2013/002174 filed Jul. 23, 2013, which claims priority of German Patent Application 10 2012 014 469.5 filed Jul. 23, 2012, the entire contents of which are incorporated herein by reference.

### FIELD OF THE INVENTION

The invention relates to the creation of an experimental plan and to the performing of a series of measurements, comprising the determination of operating data of a vehicle drive device to be tested by means of an automated statistical experimental plan. A statistical experimental plan is also referred to as Design of Experiments, hereinafter abbreviated as DoE.

### BACKGROUND OF THE INVENTION

Conventionally, technical systems are described by means of an influencing variable and a disturbing variable acting on a product or a process. This action results in measured variables that usually describe characteristics. These measured variables are then compared with target variables. Such a systemic connection can be illustrated, for example, by corresponding diagrams, such as multiphase diagrams, operating diagrams, characteristic diagrams and the like.

Methods for the examination of the behavior of technical systems can generally be subdivided into-analytic methods in which a theoretical derivation or calculation is performed on the basis of physical principles. On the other hand, empirical methods are used in which parameters are changed during the performing of experiments or in a computer simulation and an analysis is performed on the system behavior in the event of a change.

Performing an experiment requires time and resources corresponding to the necessary effort. Therefore, it is known to use a DoE, i.e. a statistical design of experiments, to determine, as precisely as possible, a cause-effect relationship between influencing factors in the form of variables as independent as possible and target variables in the form of dependent variables, using as few experiments as possible.

At the beginning of a DoE, a type of experiment is designed, for example, which describes the screening of important influencing variables with respect to their effect. The experimental results of such a design can be evaluated. Based on the evaluation, optimization plans are developed, performed and evaluated. Using statistical evaluation programs, for example, it is possible to optimize one or a plurality of target variables, for example by means of algorithms. This means that control variables can be found for process parameters, for which the target variables are at an optimum.

EP 2 088 486 A1 describes the creation of a global model of an output variable of a non-linear dynamic real system in the form of an internal combustion engine or a powertrain or of a subsystem thereof. The global model covers the entire range of all operating points of the system. It is described to perform a measurement of the system for a subset of variation points, which variation points are defined by a set of parameters of the system. In order to allow a fast and targeted development of experimental designs, as well as their global optimization under consideration of the experimental limits and other criteria, at least two variation point subsets, selected in dependence on each other, are determined successively, a common experimental design is established under consideration of the variation points of all subsets, and the system is measured based on this experimental design.

DE 199 08 077 A1 describes the application of a statistical design of experiments, in which a target variable-optimized parameterization of control variables is performed at a predetermined operating point. For this purpose, setting parameters such as ignition, lambda value, EGR mass flow and valve control times are optimized at an operating point by means of a mathematic polynomial model of the second order at most and under consideration of reciprocal dependencies of the first order, the optimization being made based on a predetermined operating point.

DE 198 19 445 A1 proposes to achieve a reduction in measuring effort by using statistical evaluation and calculation methods to reduce measurement value recording efforts in order to obtain information about a target variable. For this purpose, square polynomials are established for such target variables as nitric oxide emission NOx and soot emission in the form of the soot level, in which polynomials the influencing factors such as the start of fuel injection, the mean charging pressure, the exhaust gas recirculation rate, the air temperature in the intake pipe upstream of the inlet valve and the torque are represented in the form of terms. For example, such a square polynomial includes 21 free parameters corresponding to the polynomial coefficients. Further, it is proposed to perform a normalizing transformation into the polynomial coefficients. It is intended to thereby better represent the respective influence of the individual polynomial coefficients on the target variable result.

It is an object of the present invention to provide a design of experiments for a vehicle, which is improved with respect to prior art.

### SUMMARY OF THE INVENTION

This object is achieved with a design of experiments and the performing thereof having the features of claim 1. Further advantageous embodiments and developments are defined in the respective dependent claims, wherein both the following description and the Figures provide features and embodiments of the invention in a detailed manner. In particular, it is also possible to complete and/or replace one or a plurality of features of the independent or the independent claims with one or a plurality of features of the description. Therefore, the wording of the independent claims is a first attempt at putting the invention into words. Moreover, it is possible to combine one or a plurality of features of respective different embodiments of the invention into further embodiments of the invention.

The invention proposes the creation of an experimental design and the performing of a series of measurements, comprising the determination of operating data of a vehicle drive device to be tested, the determination being made by means of an automated statistical design of experiments (DoE). The design of experiments comprises at least the following steps:
identifying one or a plurality of target variables to be observed by the drive device in a test operation, and limiting relevant values of the one or the plurality of target variables,
assigning one or a plurality of control variables of the drive device to the one or the plurality of target variables, and
creating the design of experiments in an automated manner, based on at least two of the target variables to be observed.

Performing a series of measurements is to be understood in particular as performing an experiment according to the design of experiment in its entirety. However, it is also possible to perform an experiment in a divided manner, after the assigning and the further steps of the proposed method have been performed. For example, the design of experiments may include different ranges, for example a high load range, hereinafter referred to as high load, and a low load range, hereinafter referred to as low load. The relevant performing of experiments may, for example, be carried out independently of each other and the respective partial results may be combined with each other at a later time. Combining the design of experiments and the performing of experiments is advantageous in that it is possible to adjust the one to the other. As will be explained hereunder in more detail, this proceeding makes it possible to define target variable values and limits to be set in the DoE plan, since these are set in this manner during the performing of the experiment. Accordingly, this may also be referred to as a target variable range-specific DoE, preferably in contrast to known methods that start from a control variable range-specific DoE. DoE is the abbreviation of the English term Design of Experiments. This term refers to the above mentioned statistical design of experiments, but also to the performing thereof, wherein the design of experiments, as well as the performing is each preferably carried out in an automated manner, in particular in a manner coupled with each other.

According to a development, it is provided that the establishment of an experimental design is carried out as follows: a range of values is predetermined for a target variable, respectively, where a value of a respective target variable may only be within this range of values, and the design of experiments is established only thereafter, based on the respective range of values of the target variable, wherein the design of experiments avoids setting control variables that would lead out of the respective range of values of the target variable.

Another embodiment provides that a range of values is predetermined for a target variable, respectively, where a value of a respective target variable may only be within this range of values, and the design of experiments is established only thereafter, based on the respective range of values of the target variable, wherein the design of experiments provides setting control variables having a resolution that is lower by at least the factor of ⅕ for such control variable values that would lead out of the respective range of values of the target variable.

Thereafter, the experiments are performed based on the design of experiments. For example, it may also be provided that, while the experiments are performed, the design of experiments is further adapted and adjusted, e.g. if it is found that some of the target variable values will be in ranges outside the predetermined range of values, in particular the space of values of one or a plurality of target variables.

The vehicle drive device to be tested comprises at least one torque generator by means of which the vehicle can be moved. The torque generator may be an internal combustion engine, for instance in the form of a reciprocating piston engine. It may also be a combination of an electric motor and an internal combustion engine. The torque may also be provided exclusively via an electric drive. Further, a fuel cell or another energy generator may also be part of the drive device.

Preferably, the vehicle is a land vehicle in the form of an automobile, an agricultural machine or a truck. However, it is also possible to test the motive engine of a train. Likewise, the vehicle may be an aircraft, for instance a propeller plane, a light plane, a helicopter or even a drone. The vehicle may as well be a boat or a ship, a submarine or another watercraft. Further, other vehicles that preferably have a reciprocating piston internal combustion engine can be tested with respect to their drive device in the manner provided by the invention.

By identifying and limiting the target variable, the range is predetermined in which the results of the target variable are intended to be during the experiments and tests. Thereby, the range or space is eventually defined in which the experiments are actually performed in order to obtain a sufficient data collection. A limitation can be realized, for example, by predetermining an emission limit. For instance, it is possible to predefine a NOx emission, a CO2 emission a soot emission, e.g. in the form of a soot level, but also a sound emission. Values that exceed these predefined limits for the respective target variable are not required for the data collection. It may be provided that ranges above or below such values of the target variables are also captured. However, this is preferably effected only at a much lower resolution of experiment values than within the defined range or the defined space.

Assigning one or a plurality of control variables of the drive device with one or a plurality of target variables is achieved by means of mathematic modeling, for example. However, it is also possible, for example, to determine this relationship by means of tests on a test stand. In this context, a simple control circuit can be used, for example. By changing one or a plurality of control variables to obtain one or a plurality of predetermined target variables, it is possible to determine a relationship, e.g. in the form of a mathematical formulation. For example, one or a plurality of control variables assigned to a target variable is changed until the results are within the target variable space. This is effected preferably for different assignment pairs of control variables and target variables by means of respective controllers, preferably in an automated manner. Thereby, values of control variables or value ranges and/or combinations of values or value ranges of control variables can be ignored in the design of experiments, if it is known that the same would otherwise result in the assigned target variable being outside a parameter, e.g. outside a range. Generally, any mathematical formulation can be employed for this purpose that also indicates the basic principle of the controller—be it a P-, I- or a D-controller or combinations thereof, such as a PI-, PD-, PID-controller.

The assignment may for example include a one-to-one assignment of a control variable to a target variable. However, it is also possible to assign two or three control variables to one target variable. At least, each target variable has one control variable assigned thereto. Preferably, more than two target variables with the respective assigned control variables are eventually taken into consideration in the design of experiments, specifically more than three target variables. In this manner, a plurality of target variables may be examined and a target variable-based DoE can be performed. Further, different assignments may be provided for different sub-ranges. For example, a high load range may have another assignment among target variables and control variables that a low load range.

For example, it is provided that one or a plurality of the assignments hereunder can be used entirely or in part, the assignments being only exemplary and should not be construed to be limiting.

| Control variable | Target variable |
|---|---|
| Air mass | NOx |
| Air mass | CO2 |
| Rail pressure and/or injection pressure | Maximum conversion rate |
| | and/or noise |
| Start of injection of main injection | Position of the 50% |
| | conversion point αx50 and/or |
| | efficiency |
| EGR valve position, air mass | NOx |
| Wastegate position at the turbocharger | Engine flushing gradient Δp |
| Outlet: phase shifter position with variable | Inner temperature t of cylinder |
| outlet valve position and/or multi-part | at the time “inlet closes” |
| EGR concept: EGR distribution | |
| between high and low pressure EGR | |

These assignments are preferably used for respective controllers through which the experiments can then be performed in particular on a motor test stand.

After the assignment and the determination of the relationship between the control variables and the target variables, the design of experiments is created in an automated manner, based on at least two target variables to be observed. Here, the two target variables and their respective limits can result in a range or space within which the results of changes to the set values should or may fall.

The above proposed proceeding makes it possible, in particular, to develop global or sub-global emission models for engine calibration with reduced measuring effort. In this context, a global model is understood as the entire engine characteristic map, while a sub-global model is to be understood as a subset of the entire engine characteristic map, for example a high load range. In particular, such a design of experiments makes it possible to also include the load and the rotational speed as control variables and thus as parameters, so that a global DoE becomes possible in this manner.

An embodiment of the invention provides that the same is used in an application for a control device of an internal combustion engine. Variables presently used, for example, such as air mass, start of injection and charging pressure, have no direct connection to actual target variables such as NOx emission and consumption, in particular. However, these variables may be used as control variables if the measurement is performed on a test stand, based on such target variables as emission, consumption or noise. Thereby, the effort of performing a DoE can be minimized successfully. This will be explained in more detail hereunder.

It is intended to optimize a design of experiments for an application. It has been recognized that in a previous design of experiments using the DoE method all variable experiment parameters, such as injection quantity and injection time, as well as air quantity as a function of rotational speed and load, are quite schematically planned, within certain limits that are considered useful, and scanned completely. Many non-relevant points are addressed specifically in the corners of the normally rectangular experimental design characteristic maps for each variable parameter. These experimental points result, for example, in excessive consumption and/or excessive concentrations of pollutants and cannot be used for the application. The method proposed avoids these unnecessary characteristic map points by means of a design of experiments that is based on the target variables, so that considerable time saving is possible from the beginning.

It is therefore proposed to select characteristic maps to be scanned not on the basis of the previously usual free parameters, but based on the target variables such as consumption, concentrations of pollutants etc. The limits for these target variables are often already predefined, e.g. by legal limits or target limit values defined by the user of the internal combustion engine. Thus, there is practically no space left in the target variable characteristic map for superfluous measuring points, unless a target variable cannot be reached. The proceeding provides to first retain only the basic characteristic map of rotational speed over load as free parameters, but to automatically optimize the remaining free parameters during the experiment by means of an application routine, such that the required target variables according to the target variable DoE planning are obtained at each measuring point. The associated free parameters are then applied to the predefined target variables.

In a next step it is provided to generalize the optimization of the target variables. For this purpose, the DoE experimental planning includes, among the target variables, e.g. the attainability, and, among the free parameters, the selection of rotational speed and load points. Thereby, not all control variable ranges are applied as before. Rather, only such ranges are applied in which the target variables are met. It is a particular advantage that the determination of the experimental space limit is facilitated. Thus, the DoE experimental planning is based on target variables.

In order to perform such a DoE measuring that is oriented to the target variables, a test stand for the drive device is used, for example. Preferably, this is a thermodynamics test stand, which means that inflowing and outflowing mass flows, temperatures, power outputs and others can be detected in order to establish balances and allow other evaluations. The measuring technology on this test stand for example provides sensors for detecting exhaust gas emissions, sound emissions, mass flows, pressures and temperatures at different positions of the drive device. It is further possible to access at least one control device of the drive device and/or a series of output stages, which output stages may drive different actuators of the drive device. Further, a plurality of controllers is provided that adjust the control variables according to predefinable target variable parameters. The controllers may be integrated in the test stand, for example. However, they may also be integrated, in addition or as an alternative, in a rapid control prototyping system. This may include a dynamic description of the system to be automated and its modeling, the regulation and control design in the model, the implementation of the regulation and control design in the control device, the testing of the solution in a pure simulation environment and/or on the real system.

After one or a plurality of control variables of the drive device is thus assigned to and related to one or a plurality of target variables by means of e.g. at least one control circuit, where the preferred target variable is a NOx emission and a CO2 emission of the vehicle, in particular together with the consumption of the vehicle as a further target variable, the DoE or DoEs can be established on the basis of the correlations determined.

The drive device may then be tested on a test stand. The measured values to de determined according to the experimental planning of the DoE are based on target variables such as emissions, consumption or sound, and, in addition or as an alternative, also on process-relevant parameters such as the position of the 50% conversion point or the maximum conversion rate. The target space is thus filled with measuring points which are limited to the predefined range or space.

A further enhancement of the design of experiments is achieved if the target variables are described relative to a base. In this context, a base should be understood as a starting point in the target variable range, which may be a center point, for example. Among these, in a target variable space, a measuring point arranged between two stages or levels is provided in addition to one or a plurality of measuring points on the respective stage or level. Thus, it becomes possible to ascertain a non-linearity in tests on two stages or levels of the target variable range or space. If a target variable is examined on two stages and at the center point, a possible non-linear connection in the form of a curvature between the target variable and the control variable may be detected. In addition, it may further be determined during the statistical evaluation, whether the center point is significant. In this case, at least one target variable has a non-linear influence.

Regarding the basic proceedings of the design of experiments, the performing thereof, as well as the modeling and evaluation, reference is made to the abovementioned EP 2 088 486 A1 and in particular to the further prior art indicated for these individual matters, as well as to the publications listed in the Search Report attached to this document.

In the following, the modeling shall be described in more detail, the modeling being a requirement for the creation of a design of experiments. A modeling which uses the target variables to be observed, is realized for example with the help of mathematical functions such as polynomials, splines, wavelets, a neuronal network, e.g. including a radial basis function and/or using physical principles.

For nominal, i.e. for example categorical or also qualitative target variables, evaluation is preferably performed using variance analysis. For quantitative, i.e. for example metrical target variables, evaluation is preferably performed using regression analysis. Thus, it is possible to use regression models that are based on a linear combination of basis functions:

Linear model without interactions: y=a_{—}0+a_{—}1x_{—}1+a_{—}2x_{—}2+a_{—}3x_{—}3 for three target variables

Linear model with interactions: y=a_{—}0+a_{—}1x_{—}1+a_{—}2x_{—}2+a_{—}3x_{—}3+a_{—}4x_{—}1x_{—}2+a_{—}5x_{—}1x_{—}3+a_{—}6x_{—}2x_{—}3 for three target variables

Square or cubic models with interactions: y=a_{—}0+a_{—}1x_{—}1+a_{—}2x_{—}1̂2+a_{—}3x_{—}2+a_{—}4x_{—}2̂2+a_{—}5x_{—}3+a_{—}6x_{—}3̂2+a_{—}7x_{—}1x_{—}2+a_{—}8x_{—}1x_{—}3+a_{—}9x_{—}2x_{—}3 for three target variables

These models can be understood as Taylor expansions up to a degree of n=1 or n=2, respectively. The model parameters a_i are determined such that the deviations between the data and the model are as small as possible. For example, it is also possible to minimize the sum of squared deviations.

If the actual form of the functional correlation between target variables and control variables is known, it is possible to adjust the control variables in this function with non-linear regression.

Thus, DoEs can be established that take the following into consideration:
the number of the target variables to be examined, preferably more than two, in particular more than five, e.g. six
the type of the target variables to be examined
existing information about the correlation between target variables and control variables due to modeling
desired accuracy/reliability of the statements, e.g. on the significance of the individual results.

According to a development of the method it is provided that a calibration of the modeling is performed prior to establishing the design of experiments. The accuracy can be increased by the calibration. For example, a non-optimized calibration of an engine may be used for calibration purposes. The same can be used as a basis in the experimental space. For global DoEs, the target variable-based limits defined with respect to time and load are possibly very wide. Therefore, it may be useful to define these limits with respect to a basis point of the target variable. This facilitates the subsequent design of experiments. Further, subdividing the global DoE can be useful in particular when different behaviors of target variables are determined in different sub-ranges. For example, a NOx emission under high load conditions can be influenced by other control variables than a NOx emission under low load conditions. Therefore, for example for emission-dependent applications, a sub-global DoE related to high load conditions and a sub-global DoE related to low load conditions are established.

After the performing of the experiments and the determination of the measured values, the measured values may be validated. For this purpose, the values obtained can be matched against those that have been obtained in further tests on the test stand for purposes of comparison. For example, it may be provided that, based on the defined model structure, a determination of the essential variables and the validation, it is found that the model structure has to be modified.

Another embodiment of the proposed creation of a design of experiments provides for a transfer on other set values. This transfer can be made, for example, on the maximum conversion rate, the temperature at the time “inlet closing” or on any other set value. In particular, a transfer into a control device of an engine is also provided.

According to another idea of the invention, it is provided to use measuring results, which are obtained by means of the method proposed, in designing a vehicle application. Specifically, it is possible to thereby provide data for a control device, preferably an engine control device.

Further, according to another idea that may be independent of the above and the following aspects, but may also be correlated with these, a test stand is provided which comprises the following:
a drive device to be tested,
a measured value recording and measured value evaluation,
a respective predefined value range for one target variable, respectively, wherein at least two target variables are predefined which belong in the group comprising the NOx emission, the CO2 emission, the consumption of the internal combustion engine and the soot particle emission,
at least one control circuit which provides for the correlation of one or a plurality of control variables of the drive device with one or a plurality of target variables, wherein values of the target variable are predefined as set values of the control circuit, and
including an implemented creation of a design of experiments and performing according to one of the above and/or the following ideas or features.

The drive device preferably is a vehicle drive device which for instance comprises an internal combustion engine and/or an electric drive. The measured value recording device comprises at least one or a plurality of sensors and one or a plurality of associated data lines by means of which actual values of parameters of the drive device can be detected. Preferably, actual values of the target variables can be recorded with the measured value recording device. Thus, it is possible, e.g. while carrying out the test series, to perform a check at any time, based on the target variables acting as the set values for the control circuits used. Preferably, the measured value recording device is part of one or a plurality of control circuits used in performing the design of experiments. The measured value evaluation device comprises at least one memory in which, for example, values detected by the measured value recording device can be stored in a retrievable manner. The memory may, for example, comprise one or a plurality of areas in which different data can be stored or from which they can be retrieved. For instance, a first memory area may contain the experimental design, while a second memory area may contain values determined with respect to the experimental design. It is also possible to provide different memories therefor, as well as separated memories or memories that are integrated with each other. Preferably, a CPU is connected with the memory. The former makes it possible to detect and evaluate recorded measured values and to thereby establish a characteristic data map. Further, the CPU or also another CPU can be used, for example, to perform the experimental design in an automated manner. Further details on possible components of the test stand are evident from the above and the following description and the associated Figures.

Further, a computer program product is provided which has program code means stored on a computer-readable storage medium for performing a method as described above, when the program is executed on a computer. Thereby, it is possible, for example, to retrofit existing installations with this proceeding. Thus, already existing test systems can retroactively be enabled, by means of an update, to create and perform a design of experiments as provided by the invention.

### BRIEF DESCRIPTION OF THE FIGURES

Further advantageous embodiments and developments are evident from the following Figures. The following Figures illustrate an embodiment of an internal combustion engine with a generator. However, the details and features evident from individual Figures are not restricted to the respective Figure or embodiment. Rather, one or a plurality of features can be combined with one or a plurality of features from various Figures, as well as with features from the above description to from new embodiments. In particular, the statements hereunder do not serve as limitations of the respective scope of protection, but explain individual features and their possible interaction. In the Figures:

FIG. 1 is an exemplary illustration of an internal combustion engine to be tested,

FIG. 2 is a schematical illustration of a proceeding according to the prior art,

FIG. 3 is a schematical illustration of a proceeding as provided herein,

FIG. 4 is a schematical illustration of a transfer on global DoEs,

FIG. 5 illustrates an example of a result space,

FIG. 6 illustrates an example of a modeling which uses a radial basis function,

FIG. 7 illustrates a validation,

FIG. 8 illustrates a first flowchart,

FIG. 9 illustrates a second flowchart for performing the method proposed, and

FIG. 10 is an exemplary illustration of a controller used.

### DETAILED DESCRIPTION OF THE INVENTION

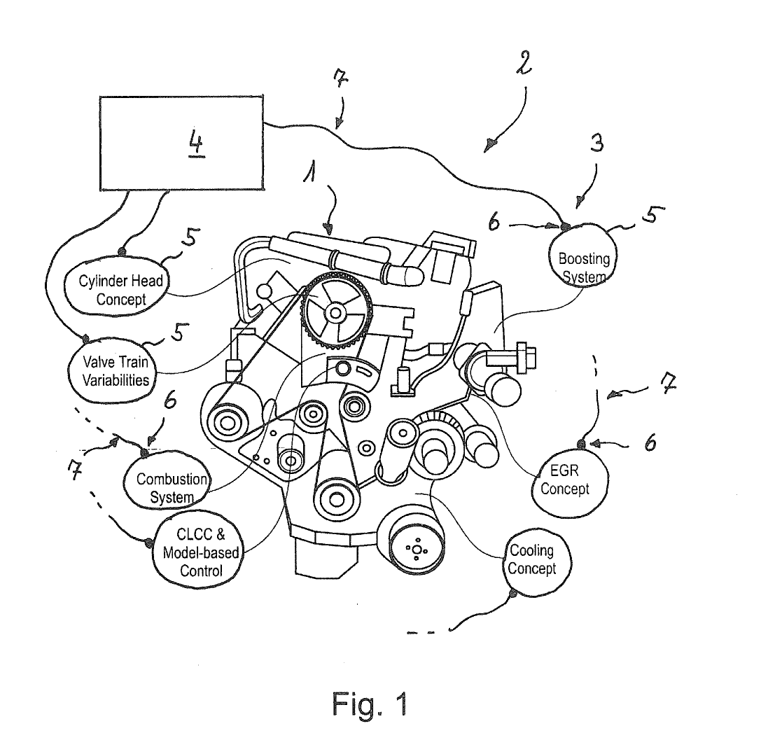

FIG. 1 is an exemplary illustration of a tested internal combustion engine with different schematically illustrated areas which, during the development, are each developed with consideration to a specification sheet and which, in combination as an overall concept, at the same time have to meet the defined primary requirements. The cylinder head concept, the valve train system, the internal combustion system, the cooling concept, the exhaust gas recirculation concept and the booster system must be designed and tested. This may be very extensive with respect to necessary measured values and the test stand tests required therefor. Conventionally, the series of measurements must be very large, timeconsuming and expensive. Currently, directly accessible control variables are used in DoE plans, such as air mass, SOI, etc. Target ranges can only be covered with the use of complex constraints on the input variables, or the target space can only be covered in a limited manner. Preferably, the internal combustion engine is arranged as a drive device 1 in a test stand 2. Besides the drive device 1, the test stand 2 comprises at least one measured value recording device 3 and a measured value evaluation device 4, each illustrated schematically. The measured value recording device 3 and a measured value evaluation device 4 may be designed such that they are adapted to the respective target variables and thus to different areas 5 of the drive device 1. As illustrated schematically and exemplary, the measured value recording device 3 extends from each of the encircled areas 5 of the drive device 1 to the measured value evaluation device 4, each area 5 for instance comprising one or a plurality of sensors 6. Signals, such as measuring signals, but also control signals for actuators, can be supplied to and, if so desired, also from the respective areas 5 via the data lines 7 indicated.

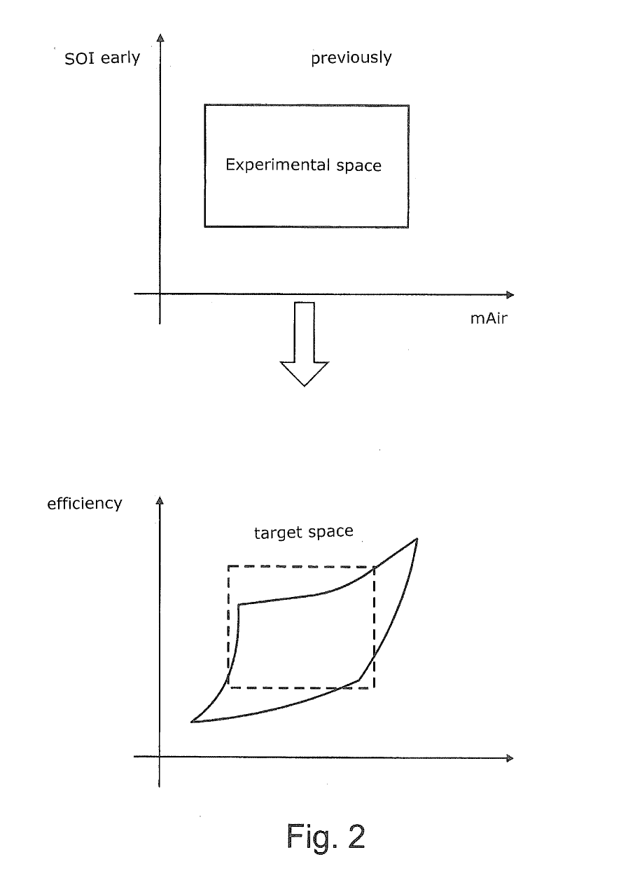

FIG. 2 shows the presently known prior art regarding the use of DoEs. Here, the starting point is the experimental space. The same may be limited by the fact that rotational speeds or velocities are outside the intended field of application of the vehicle. The experimental space is defined essentially by the control variables.

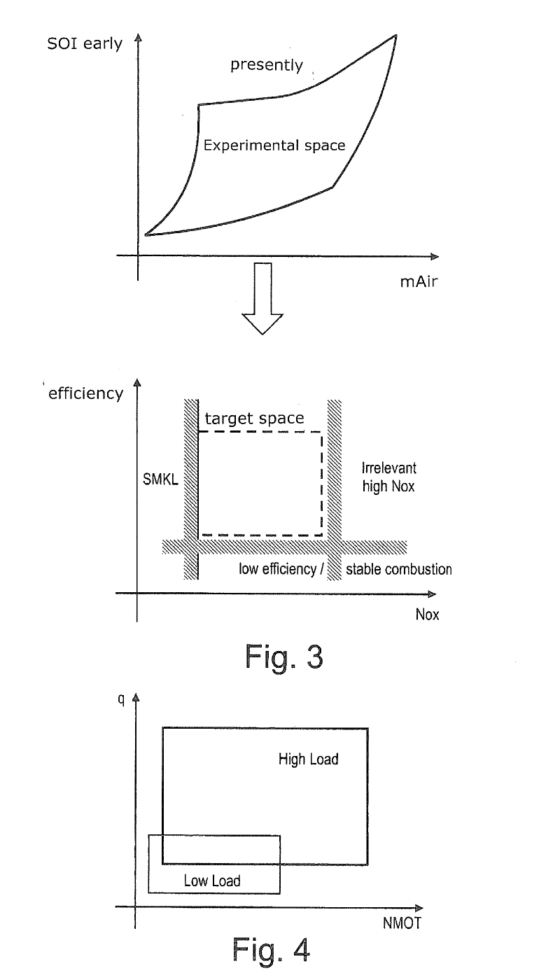

On the other hand, FIG. 3 illustrates the proceeding as provided. Here, the target variables are the starting point and the control variables are varied in the experiment such that the target variables are reached. Thus, a target variable space is predefined which the control variables must observe in the event of a change. In this manner, the experimental space is limited to a size in which the results of experiments carried out actually lie in the target variable space, are intended to lie in or are at least close to the same. In this regard, instead of air mass, SOI, etc., physically relevant inputs such as q, αX50, log(c(NOx)), TCyl, dp, rEGR may be used in the form of control variables.

Wherein:

| | |
|---|---|
| αX50 | Position of the 50% conversion point |
| log(c(NOx)) | NOx concentration at an optional or a |
| | selected point along the exhaust gas |
| | system |
| TCyl | Temperature in the cylinder at the time |
| | “inlet closes”, generally not measured |
| | but modeled |
| Dp | Engine flushing gradient |
| rEGR | EGR mass distribution |
| SOI | Start of injection of main injection |
| Q | Injection quantity, corresponding to |
| | load |

Here, the NOx concentration for example is used in a logarithmic form. It has been found that, in evaluations, when different levels of high or low concentrations exist in different areas, a presentation can advantageously be made in this manner of presentation. The same is true for other target variables and their distribution of values due to an experimental design performed. For example, different mechanisms, and thus control variables, may be an indication that a logarithmic presentation of the results for a target variable may be advantageous. With regard to a NOx concentration, this is advantageous, for instance, if a low load range and a high load range are considered together. In the low load range, the NOx concentration differs from that in the high load range, which, if presented together, could otherwise not be illustrated as clearly in a diagram.

Besides being used within the framework of local DoEs, the method proposed can also find application in the series calibration of control devices of internal combustion engines. In this context, local DoEs are DoEs that each have one load point and a special rotational speed within which the target variables can be controlled by the control variables in the predefinable range or space. Thus, local DoEs are restricted to one load point. The method proposed has the advantages of a significant simplification of the determination of the experimental space, a better filling of the target space and a limitation of the experimental space to the actually useful ranges. FIG. 3 illustrates the connection between the experimental space and the target space. The arrow in the Figure is directed opposite to that in FIG. 2. The target space is defined by limits such as, for example, an emission limit for NOx, for example, with NOx being one of the target variables. Further, a limit of the target space or the target range can be predefined by an instable combustion, a knocking, a power output that is too low, and also by the smoke limit, abbreviated as SMKL.

As the experiments are performed, the control variables may also reach target variable values that are outside the experimental space. Due to the approach according to which preferably rectangular spaces are scanned, about 30 to 40% of the target variable values may lie outside of the predefined target variable range or space. Further limitations could be applied thereto, but this would require additional effort. Therefore, in a target variable-based DoE planning, it is also taken into consideration what effort for a further limitation of the target variables or the assignment would still be reasonable when compared to a performing without further limitation.

FIG. 4 is a schematical illustration of a transfer of one target space onto another in the form of a transfer onto global DoEs. Here, physically relevant inputs are used, such as, for example, Δp, NOx, Tcyl, rEGR, αx50 and also dmfbmax, which is an abbreviation of a maximum conversion rate of the fuel in the combustion chamber. In the experimental plan, at least two, preferably most of the variables are varied relative to a basis. This means that a basis point is used as a starting point and that the limits of the target variables are then determined relative to this basis point and the design of experiments is subsequently established with reference thereto. It is possible to subdivide the characteristic map into zones or to make a subdivision according to inputs, in particular different subdivisions may be made for various reasons. An example for this may be different objectives, but also different relevancies of target variables in different ranges of a characteristic map. For example, the CO2 emission may be relevant in one range, while in another range the NOx emission and the consumption are relevant. A subdivision is illustrated as an example, which is determined by different control and target variables:

a) Low Load with q, NMOT, rEGR, Δp, NOx, Tcyl, αx50

b) High Load with q, NMOT, rEGR, Δp, NOx, αx50

with, on the one hand, the control variables for a):
q, NMOT, dmfbmax, tiPil1, qPil1

and, on the other hand, the control variables for b):
q, NMOT, tiPil1, tiPil2, qPil1, qPil2, wherein the abbreviations of various control variables not mentioned above have the following meanings:
   q. injection quantity, corresponding to the load
   NMOT: rotational speed of crankshaft
   tiPil1: time of pilot injection 1
   qPil1: quantity of fuel for pilot injection 1
   tiPil2: time of pilot injection 2
   qPil2: quantity of fuel for pilot injection 2

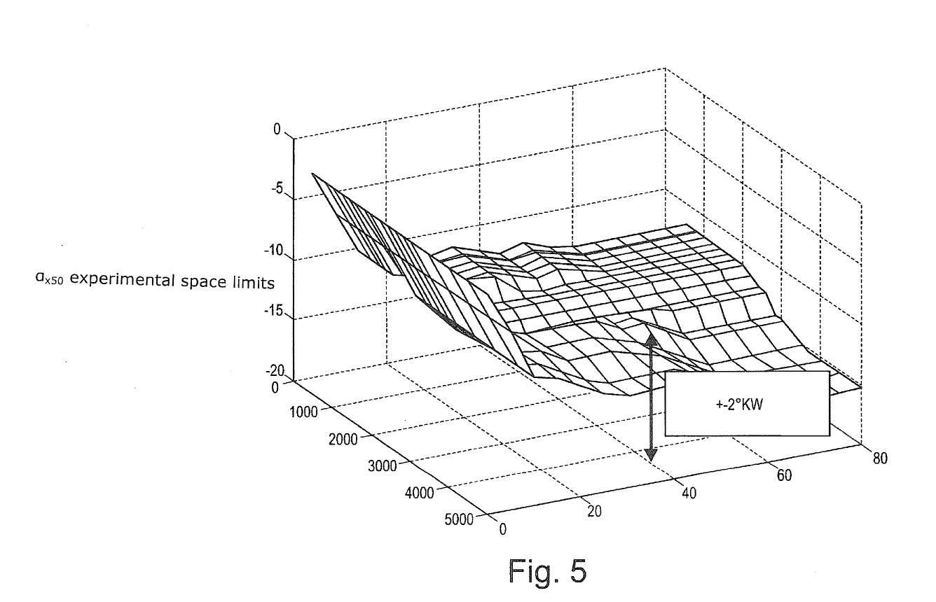

FIG. 5 is an exemplary illustration of a schematical example of a test plan, namely for the high load range, in the form of a n-dimensional DoE. A method known from the field of DoE was used herein, namely the Space Filling Design, however, with a logarithmic distribution of the NOx being made. This example indicates:

135 points including 3 stabilization points, 5 repetition points, 8 validation points for a rotational speed range Of 1200-2800 rpm and an injection quantity of 10 to 50 mg/stroke. The relative experimental space limits for all set values—except for the rotational speed n and the injection quantity q, qPil, tiPil—are based on a basic calibration.

For a relative modeling of the changes, each DoE point should have an associated basis point. The basis point is the selected rotational speed/load point from the basic data set without changes. Should it be necessary, e.g. because of slow conditionings, to sort the experimental design e.g. with respect to the charge air temperature, this should preferably be done in a V- or W-shape, i.e. ascending and descending, so as to be able to subsequently apply drift corrections. Arranging the experimental design in an ascending and descending manner, i.e. in a V- or W-shape, may also be carried out in reverse order, depending on the beginning of the experimental design. This may also find application under different conditions and is not only applicable in cases of slow conditioning. In particular when a drift correction is made, the use of descending and ascending values, or vice versa, is preferred.

It has been found that a model based on physical inputs and functions makes it possible to represent global relationships. However, linear models based on conventional inputs, such as the air mass or SOI, for example, have shown that the accuracy of the model may possibly be useless and that, further, tendencies may be represented in a wrong manner.

An optimization of the physical modeling allows an engine performing to be represented well in the manner proposed. Here, all models can be extrapolated by default. In one embodiment, no physically absurd values can occur, e.g. a soot level SZ<0. Further evaluations of the measured data and the models may be used for model development, on the one hand, and, on the other hand, for the optimization of the provision of data, as well as for control device functions in vehicles.

A global optimization is also possible. For this purpose, it is useful, for example, to provide for the use of a so-called tool box. According to a development, it is provided to reduce the optimization to an optimization of consumption with constraints.

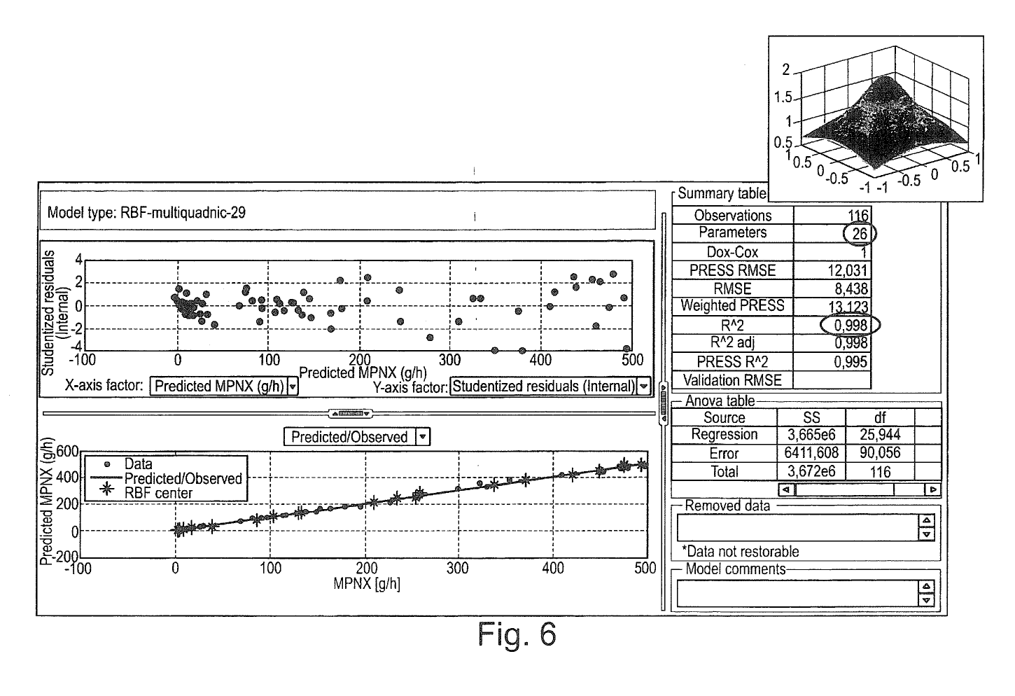

In this regard, FIG. 6 shows an example of a modeling using a tool box. The modeling was performed with the help of a Matlab/Simulink tool box using non-linear models, e.g. radial basis functions. The illustration shows the advantages of a logarithmical evaluation, since concentrations that deviate significantly from each other can still be presented and evaluated together.

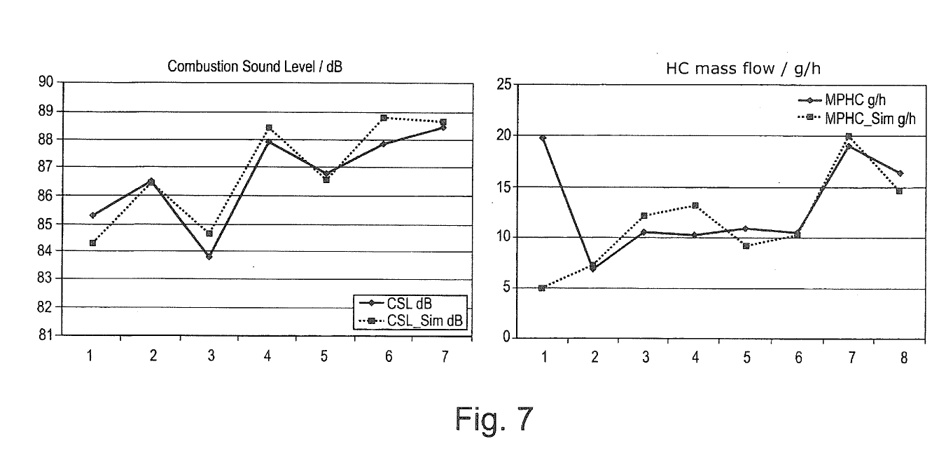

FIG. 7 shows a validation under high load, wherein the target variables were determined, on the one hand, by experiments according to the manner proposed and, on the other hand, by simulation.

First results of the method proposed show the following:
it seems generally possible to represent an engine performing;
up to the present, some experience with MBC has been gained that confirm this statement;
physically absurd values can occur, e.g. SZ<0;
according to one embodiment, no extrapolability exists. Interpolation errors that are not detected in the modeling, carry great risks due to the very flexible models;
it is also possible to represent complex connections such as pressure waves in the rail/injector;
the use of non-physical models requires the implementation of commercial tools such as Model Based Calibration Toolbox and Matlab, as well as skilled personnel;
it is possible to represent pressure waves, for example;
further optimization potential by adapted experimental designs with distinction between global and local variables, e.g. for NOx;
a non-physical modeling bears the risk of possible interpolation errors and lack of extrapolability;
also in case of non-physical modeling, it is preferred to establish and perform the experimental design based on physical variables, since the preparation effort is thus reduced significantly;
further, a logarithmical distribution of target concentrations is useful in achieving an improved model quality.

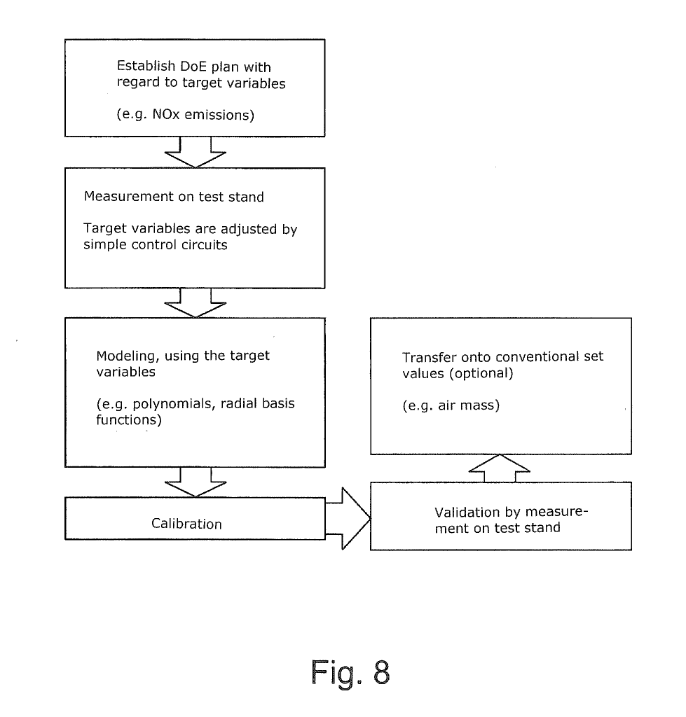

FIG. 8 shows an exemplary embodiment of a first flowchart. Here, after the DoE plan has been established by assigning control variables to target variables and by limiting the target variable space, measurements are made on the test stand. The target variables are set by means of control circuits that are as simple as possible so that they lie in the predefinable target variable space. Preferably, each control variable for each operating point is adjusted until the target variable parameters are reached. Thereafter, a modeling can be made, for example according to mathematical methods, using the target variables, while a calibration may be performed subsequently. A validation of the values thus found can be made at the test stand by measurement. Thereafter, the transfer can be made, which means, for example, supplying a parameter set to an engine control device.

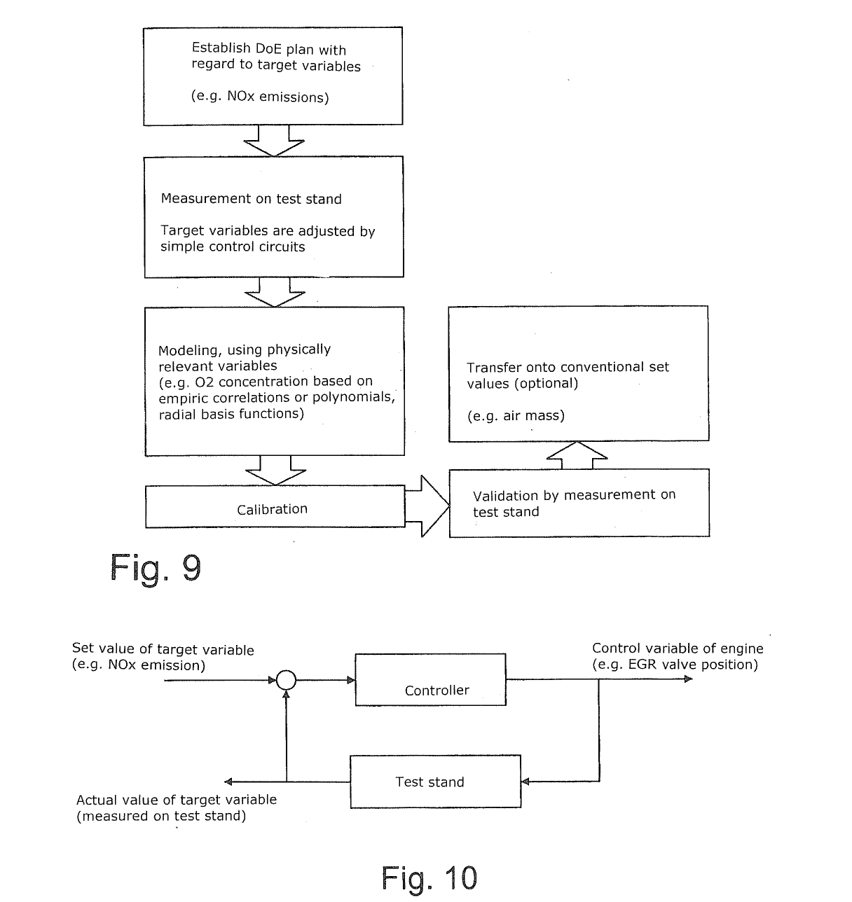

FIG. 9 shows a second flowchart. Different from the previous flowchart, the modeling is done using physically relevant variables, e.g. such variables as mentioned above and shown in the Figure. It is also possible to obtain a modeling using both possibilities illustrated in FIG. 8 and FIG. 9. In particular in the event of multiple interdependencies of a plurality of control and target variables, it is possible, in adjusting the target variables, to use multi-dimensional control algorithms, functions, characteristic diagrams or even model-based algorithms.

FIG. 10 shows a basic structure of an implementable controller, wherein the target variable is used as the set value. Such a controller is used at a test stand for the engine to be tested, so as to allow a correlation between the target variable and the control variable corresponding to the assignment. This target variable-based approach makes it possible to use only such control variables for which an actual value of the target variable lies in the predefinable target variable space. If control variable ranges exist that prevent this and automatically lead to actual values outside, these can be ignored.

1. A method for creating an experimental design and performing of a series of measurements, comprising:
determining operating data of a vehicle drive device to be tested, the determination being made by means of an automated statistical design of experiments (DoE);
wherein the design of experiments includes at least the following steps:
identifying one or a plurality of target variables to be met by the drive device in a test operation, and limiting relevant values of the one or the plurality of target variables;
assigning one or a plurality of control variables of the drive device to the one or the plurality of target variables; and
creating the design of experiments in an automated manner, based on at least two of the target variables to be met.

2. The method of claim 1, wherein a range of values is predetermined for a target variable, a value of a respective target variable may only be within the range of values, and the design of experiments is carried out only thereafter, based on the respective range of values of the target variable, wherein the design of experiments avoids setting control variables that would lead out of the respective range of values of the target variable.

3. The method of claim 1, wherein a range of values is predetermined for a target variable, respectively, where a value of a respective target variable may only be within this range of values, and that the design of experiments is carried out only thereafter, based on the respective range of values of the target variable, wherein, for target variable values that would lead out of the respective range of values of the target variable, the design of experiments provides setting control variables having a resolution that is lower by, preferably, the factor of ⅕.

4. The method of claim 1, wherein a correlation of one or a plurality of control variables of the drive device with one or a plurality of target variables is performed by means of at least one control circuit.

5. The method of claim 1, wherein a NOx emission and a CO2 emission of the vehicle are used as the target variable.

6. The method of claim 1, wherein a consumption of the vehicle is used as the target variable.

7. The method of claim 1, wherein the drive device is tested on a test stand and the target variables are adjusted in the process by means of control circuits.

8. The method of claim 7, wherein the control circuits use predefined target variable values as set values.

9. The method of claim 1, wherein a modeling is performed with the use of the target variables to be met.

10. The method of claim 9, wherein a calibration of the modeling is performed.

11. The method of claim 9, wherein a validation of the modeling is performed.

12. The method of claim 9, wherein a transfer of the modeling on other set values is performed.

13. The method of claim 1, wherein a basic characteristic map of “rotational speed over load” is first retained as a free parameter, while remaining free parameters are automatically optimized during the series of measurements by means of at least one application routine, such that the required target variables according to the target variable DoE planning are obtained at each measuring point, and that, in a subsequent step, the optimization of the target variables is generalized, wherein, among the free parameters, the selection of rotational speed and load points is included in the DoE planning, whereby only such control variable ranges are applied in which the target variables are met.

14. (canceled)

15. The method of claim 1 further comprising using the obtained measuring results in the design of a vehicle application.

16. An apparatus comprising:
a computer program product with program code stored on a computer-readable storage medium configured to determine operating data of a vehicle drive device to be tested, the determination being made by means of an automated statistical design of experiments (DoE);
wherein the design of experiments includes at least the following steps:
identifying one or a plurality of target variables to be met by the drive device in a test operation, and limiting relevant values of the one or the plurality of target variables;
assigning one or a plurality of control variables of the drive device to the one or the plurality of target variables; and
creating the design of experiments in an automated manner, based on at least two of the target variables to be met
when the program is executed on a computer.

17. A test stand apparatus (1) comprising:
a drive device to be tested (2),
a measured value recording (3) and measured value evaluation (4),
a respective predefined value range for one target variable, respectively, wherein at least two target variables are predefined which belong in the group comprising NOx emission, CO2 emission, consumption of the internal combustion engine and soot particle emission,
at least one control circuit which provides for the correlation of one or a plurality of control variables of the drive device (2) with one or a plurality of target variables, wherein values of the target variable are predefined as set values of the control circuit, and
including an implemented creation and performing of a design of experiments that includes determining operating data of a vehicle drive device to be tested, the determination being made by means of an automated statistical design of experiments (DoE);
wherein the design of experiments includes at least the following steps:
identifying one or a plurality of target variables to be met by the drive device in a test operation, and limiting relevant values of the one or the plurality of target variables;
assigning one or a plurality of control variables of the drive device to the one or the plurality of target variables; and
creating the design of experiments in an automated manner, based on at least two of the target variables to be met.
